(19) United States
(12) Patent Application Publication
(10) Pub. No.: US 2008/0160069 A1
(43) Pub. Date: Jul. 3, 2008

(54) Constricting Gel Assay and Therapeutic Patch

(75) Inventors: Andrew J. Maniotis, Berwyn, IL (US); Robert Folberg, Northbrook, IL (US)

Correspondence Address: BARNES & THORNBURG LLP, P.O. BOX 2786, CHICAGO, IL, 60690-2786, US

(73) Assignee: The Board of Trustees of the University of Illinois, Urbana, IL (US)

(21) Appl. No.: 11/911,373

(22) PCT Filed: Apr. 11, 2006
(86) PCT No.: PCT/US2006/013672

§ 371 Date: Nov. 27, 2007

Related U.S. Application Data
(60) Provisional application No. 60/670,340, filed on Apr. 12, 2005.

(51) Int. Cl. A61K 9/70; G01N 33/483; A61K 35/12

(52) U.S. Cl. 424/449; 435/40.5; 424/93.7

(57) ABSTRACT

Method and compositions relate diagnostic tools to determine invasive potential of tumor cells. Therapeutic patches include a component from the extracellular matrix seeded with tumor cells from an individual and a fibrin backing. Extracellular matrix-based constriction assays are useful to screen for anti-tumor compounds.

### BACKGROUND

Tumor cells seeded on an extracellular matrix in a three-dimensional culture, provide a diagnostic and therapeutic tool.

Current cancer detection tests include biopsies and diagnostic imaging techniques. Biopsies, although causing discomfort to the patient, can be outpatient procedures, and oftentimes either numerous samples are taken or one small portion of the tumor must be removed to ensure consistent and accurate results. During a biopsy, a portion of the patient's tumor is extracted for observation. Upon staining, the sample is examined under a microscope for evidence of abnormal cells or growth. This test cannot conclusively tell whether or not a patient has cancer; it can only tell them if the cells sampled are abnormal. Further testing is required to achieve an accurate diagnosis. In detecting breast cancer alone, it has been estimated that 1.6 million biopsies are performed each year as standard procedure.

Some of the more popular imaging techniques are Fluorescence Bronchoscopy, Gamma PET Imaging, Miraluma Breast Imaging, MRI, and Spiral Computed Tomography. Fluorescence Bronchoscopy is used to determine whether the patient has lung cancer. Commonly referred to as “blue-light bronchoscopy”, this method can diagnose minuscule in-situ tumors, which appear reddish-brown in fluorescence compared to the green tint of healthy tissue. MRI is a common tool used for its diagnostic capabilities in both cancer and other health ailments. It provides an option for high-risk patients and can detect tumors that are undetectable by the patient. Spiral CT is another form of imaging which can provide 3-D visualization of the body. This visual diagnostic tool can aid in the detection of small tumors in the lungs that may not be seen with a chest X-ray. However, imaging tends to be expensive and depending on the procedure, time consuming.

The growth of cells on gel rafts floating upon a pool of cell culture medium is a research method that is widely used for many purposes in cell biology and cellular medicine. One such application of this method is in the study of cell growth. Growth of many types of cancer cells and certain types of normal cells, most notably fibroblasts, on such rafts can cause the rafts to constrict to a considerable degree. This constriction effect is sometimes used to identify clonal populations of cancer cells within a cell population.

### SUMMARY

A tri-purpose diagnostic and therapeutic bioassay includes a simple raft of floating ECM components, e.g., collagen or thrombin plus plasminogen, that is used to (1) assess the stage of cancer when cells from a biopsy from a tumor are placed on it, (2) test the toxicity and efficacy of new chemotherapeutic compounds or regimens; and (3) introduce dried tumor response of any tumor cells within the body. Introduction may be via a dermal patch.

A convenient and complementary in vitro diagnostic test for cancer gives more conclusive information about a tumor while being only as invasive as a current biopsy procedure. The degree of constriction caused by the growth of cancer cells on a gel raft was correlated with the degree of invasiveness of the cancer cells. Also, the unexpected loss of weight of a gel raft was correlated with the degree of invasiveness of the cells growing on the raft. Visual examination of the structural patterns developed by cells growing upon a gel raft provides an additional means for the estimation of the invasiveness of cells growing on the raft. The degree of constriction, the degree of weight loss and the types of structural patterns formed when invasive cancer cells are grown on gel rafts are useful in the screening of chemical compounds as potential anti-tumor agents. A means is provided for estimating the level of anti-tumor immune system activity exhibited by a cancer patient. A dermal patch stimulates the immune system of a cancer patient to mount an immune response against tumor cells.

The bioassay includes ECM proteins and is useful in the classification of tumor cells. Cancer cells obtained from a patient's tumor were categorized by their aggressiveness according to the quantification of their constriction. As a diagnostic tool, polymers of extracellular matrix proteins are seeded with tumor cells. The gel demonstrates constriction and degradation caused by malignant tumor cells, and also exhibits perfusion of ECM channels created by malignant tumors cells through which plasma sized molecules can pass, but not red blood cells.

There are at least three methods of quantification applied to the constricted gels: area calculation during constriction, dextran perfusion of ECM channels, and dried gel weight comparisons. The area of the original gel was compared to the area of the constricted gel when seeded with aggressive cells using imaging software. The extracellular channels formed were quantified by dyeing the gels with fluorescently dyed dextrans to quantify the degree of perfusion. The invasiveness of the aggressive cells was determined by drying and weighing these gels and comparing them to control gels which are unseeded, or to controls using fibroblasts. From these measurements, cancer cell malignancy was established.

The bioassay uses a patient's own serum (fibrinogen) obtained from a blood sample and added to recombinant thrombin. Further, the dried gels (with tumor cells seeded) are used in vivo as an immunopatch by attaching the dried gel to a fibrin backing made from fibrinogen and recombinant thrombin. By placing the immunopatch in contact with exposed dermis, the immune response and possible immunorejection of that patient's own tumor cells is examined. Fibrinogen is advantageous in this extended therapeutic application of the gel because of its clotting abilities and biocompatibility.

Tumor cells that are present on ECM resist chemotherapeutic drugs. A clot made out of thrombin and fibrinogen serves as a platform for testing out new chemotherapeutic drugs. The degree of constriction of the bioassay along with using Trypan Blue (which measures cell viability) is helpful in testing the degree of the effect of new drugs on malignant cells.

Use of a matrix gel assay to determine invasive potential of tumor cells includes the steps of:

(a) providing a first and second matrix support comprising at least one component of the extracellular matrix (ECM) suspended in a cell culture medium;

(b) seeding the first matrix support with tumor cells and the second matrix support with control cells;

(c) providing conditions for the tumor cells to grow in the first matrix support and the control cells to grow in the second matrix support;

(d) preparing a dried sample of the matrix support;

(e) comparing the dried weight of the first matrix of the tumor cells to the dried weight of the second matrix of the control cells; and

(f) determining the invasive potential of the tumor cells by comparing the dried weight of the first matrix with tumor cells and the dried weight of the second matrix with control cells.

Use of a matrix gel assay to determine invasive potential of tumor cells includes the steps of:

(a) providing a first and second matrix support comprising at least one component of the extracellular matrix (ECM) suspended in a cell culture medium;

(b) seeding the first matrix support with tumor cells;

(c) providing conditions for the tumor cells to grow in the first matrix support;

(d) preparing a dried sample of the first and the second matrix supports;

(e) comparing the dried weight of the first matrix of the tumor cells to the dried weight of the second matrix; and

(f) determining the invasive potential of the tumor cells by comparing the dried weight of the first matrix with tumor cells and the dried weight of the second matrix.

The tumor cells may include melanoma cells, and the tumor cells may be seeded to a saturation density. In an aspect, the matrix support is air-dried.

Use of a matrix gel assay to screen for a potential anti-tumor agent includes the steps of: (a) providing a first and second matrix support comprising at least one component of the extracellular matrix (ECM) suspended in a cell culture medium, wherein the first and second matrix supports are seeded with tumor cells;

(b) contacting the first matrix support with the candidate anti-tumor agent;

(c) providing conditions for growth of tumor cells in the matrix supports;

(d) preparing a dried sample of the matrix supports;

(e) comparing the weight of the first matrix with the tumor cells to the dried weight of the second matrix of the tumor cells; and

(f) determining that the candidate anti-tumor agent is an antitumor agent if the dried weight of the first matrix with tumor cells is significantly more than the dried weight of the second matrix with tumor cells.

Use of a matrix gel assay to screen for a potential anti-tumor agent includes the steps of:

(a) providing a first and second matrix support comprising at least one component of the extracellular matrix (ECM) suspended in a cell culture medium, wherein the first matrix support is seeded with tumor cells and second matrix support is seeded with non-tumor control cells;

(b) contacting the first and second matrix supports with the candidate anti-tumor agent;

(c) providing conditions for growth of tumor cells and control cells in the matrix supports;

(d) preparing a dried sample of the matrix supports;

(e) comparing the weight of the first and second matrix supports contacted with the candidate anti-tumor agent to the dried weight of the first and second matrix supports not contacted with the candidate anti-tumor agent; and

(f) determining that the candidate anti-tumor agent is an anti-tumor agent if the dried weight of the first matrix with tumor cells contacted with the candidate anti-tumor agent is significantly more than the dried weight of the first matrix with tumor cells not contacted with the candidate anti-tumor agent and the dried weight of the second matrix support with control cells contacted with the candidate anti-tumor agent is similar to the dried weight of the second matrix support with control cells not contacted with the candidate anti-tumor agent.

The control cells may include fibroblasts or other non-tumor cells.

Use of a matrix support to stimulate immune response against tumor cells includes the steps of:

(a) obtaining a matrix support comprising seeded tumor cells, wherein the matrix and the cells are in a dried condition;

(b) administering the dried matrix support with tumor cells as a dermal patch to stimulate immune response; and

(c) monitoring the immune response against the tumor cells.

A therapeutic patch includes:

(a) a first layer comprising a matrix support comprising at least one component of the extracellular matrix seeded with tumor cells from an individual, wherein the matrix support is in a dried condition; and

(b) a second layer comprising fibrin and thrombin, wherein the second layer is adapted to be applied as a dermal patch.

The therapeutic includes fibrin that is derived from fibrinogen that can be obtained from a serum sample of an individual. The thrombin may include a recombinant thrombin.

The terms “tumor” or “cancer cells” are used interchangeably; “subject” and “patient” are used interchangeably.

### BRIEF DESCRIPTION OF THE DRAWINGS

FIG. 1 is a flow chart illustrating the relationships between diagnostic tools and therapeutic devices described herein (A). This flowchart shows how the bioassay serves as a diagnostic tool. A1: A biopsy is performed to obtain a subject's tumor cells to be seeded onto the bioassay. A2: shows that the bioassay is composed of extracellular matrix proteins including collagen I, fibrinogen derived from the subject, and recombinant thrombin. A3: indicates the gel is seeded with the tumor cells obtained from biopsy of the subject. After a few days, a technician observes the gel for constriction. The constriction of the gel indicates that the seeded tumor cells are aggressive; no constriction would indicate the “cancer” is really benign tumor cells. A4: after the cells are identified as an aggressive type, the gel is then dyed using dextrans with increasing weights up to 2,000,000 Daltons. In this step, the aggressiveness of the cancer cells is classified. If the technician observes dextran perfusion at levels of 70,000 Daltons or less, and little or no perfusion at greater dextran weights, then the presence of vasculogenic mimicry is confirmed. This shows that a network of extracellular matrix channels (a vasculogenic mimicry pattern) is forming in the gel, and not endothelial cell-lined vasculature. At this step, the cancer cells are classified as highly aggressive, metastatic or both aggressive and metastatic; if not, the cells are aggressively malignant. A5: is a step to dry the seeded gel in an oven. This is the preparation step for weighing the gel. The weighing step A6 is to verify the invasiveness of the seeded cancer cells. In this step, the control (unseeded) and seeded gels are weighed separately. The weights are then compared. If the weight of the seeded gel is less than the control gel, the cells are invasive; else, they are non-invasive.

FIG. 1(B) is a flowchart showing how the bioassay serves as a therapeutic device. B1: indicates that the effectiveness of drugs can be tested using gels seeded with aggressive tumor cells. Because gel constriction is observed when in contact with aggressive tumor cells, drug effectiveness can be measured by the blocking of gel constriction. Trypan Blue can also be used to determine the effectiveness of a drug on tumor cells that are seeded on the gel. B2: indicates an immunopatch made using the dried gels attached to an adhesive fibrin backing made from the subject's fibrinogen. In B3: the tumor cells are reintroduced into the body by attaching the immunopatch to a subject's dermis. In B4, cells are reintroduced into the body via the immunopatch to study immune response and immunorejection of a patient's tumor.

FIG. 2 shows gel raft contraction in the presence of invasive tumor cells.

FIG. 3 are photomicrographs illustrating cell structure morphologies of normal, invasive and highly invasive/metastatic cells grown on thick rafts of ECM. FIG. 3A shows a monolayer of normal cells; FIG. 3B shows the cord-like structures formed by invasive cells. FIG. 3C shows the looping patterns present in nests of highly invasive cells.

FIG. 4 illustrates an assessment of anti-tumor agents using a gel constriction assay. The left pair are BJ19 normal human fibroblasts exposed to an experimental anti-tumor compound. Constriction of the rafts indicates that the experimental compound exhibits minimal cytotoxicity toward normal cells. The right pair are C918 primary melanoma cells (highly invasive) exposed to the same experimental anti-cancer compound. Lack of constriction indicates that the invasive character of these cells has been suppressed by the experimental compound.

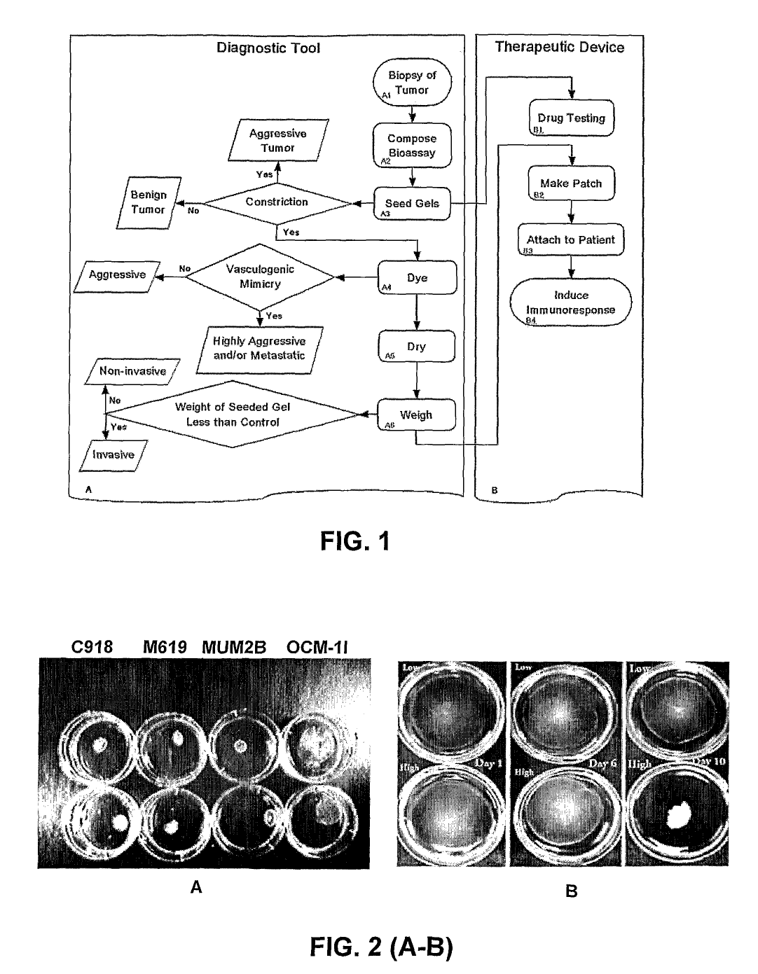

### DETAILED DESCRIPTION

Interactions between tumor cells and the extracellular matrix (ECM) are factors in the behavior of tumor cells in-vivo. In particular, invasive cancer cells are capable of remodeling the ECM. Such remodeling of the ECM can be correlated with both the ability of the tumor cell to migrate or metastasize within the body and the resistance of the tumor cells to anti-tumor agents. Tumor cells were grown on rafts comprised of gels consisting of ECM proteins in a manner that correlates with clinical parameters and events. This correlation permits cells grown on ECM gel rafts to be used as surrogates for the assessment of the degree of invasiveness of tumor cells; the immune status of a cancer patient; and the sensitivity of the tumor cells to therapeutic agents that are essential components in the development of a clinical treatment plan for a cancer patient. These same correlations also provide an efficient means for initially screening chemical entities as potential anti-tumor agents and as agents that suppress the invasiveness of tumor cells.

The following examples are embodiments of the present disclosure and are not intended to limit the scope of the disclosure.

### EXAMPLE 1

### The Use of an Unconstrained Gel Matrix to Assess the Invasiveness of Tumor Cells by Visual Inspection

Unconstrained collagen gel matrices were prepared by gently layering 250 μl of Type 1 collagen at 3-10 mg/ml (B&D Biosciences, Oak Park, Mass. 01730) in on top of 3 ml of EMEM medium (BioWhittaker, Inc., Walkersville, Md.) supplemented with heat inactivated 15% fetal bovine serum (FBS, Fisher, Ontario, Canada) in a well of a 6 well (6×35 mm) plate. No exogenous extracellular matrix (ECM) proteins or growth factors were added to the medium. Gel matrices may be prepared in a similar manner from Matrigel®, gelatin, fibrin and other gel-forming proteins, either singularly or in combination. Fibrin and fibrin-containing gels are most conveniently prepared by layering a solution containing fibrinogen plus thrombin on top of the medium and forming the fibrin in-situ. The plates containing the floating gel layers were then heated for 30 minutes at 37° C. in a humidified incubator to polymerize the gels. Rafts comprised of other extracellular matrix (ECM) proteins such as laminin and fibronectin; synthetic ECM materials such as MatriGel®; or gel forming proteins such as gelatin and fibrin may be prepared in a similar manner. The preferred approximately circular gel rafts of approximately constant thickness are most effectively prepared if the collagen solution is dispensed on top of the EMEM medium at a constant flow rate of approximately 250 ul/sec) and the viscosity of the collagen matrix solution is adjusted as necessary to give a well defined edge margin without spreading to the boundaries of the well in which the raft is formed.

The invasiveness of tumor cells is assessed by plating tumor cells at a supersaturating cell density of approximately 1×10⁷ on a gel raft prepared as described herein and incubating the cells under the appropriate conditions for a sufficient period of time for cell growth. Rafts seeded with primary uveal melanoma cell lines of low (OCM1a) and high (M619, C918) invasive potential, and a highly invasive metastatic uveal melanoma cell line (MUM2B) were incubated at 37° C. in a humidified incubator under an atmosphere of 5% CO₂/balance air for a period of 10 days. Gel rafts seeded with normal human fibroblasts and unseeded gel rafts were used as controls. The resulting grown cells were then examined visually to determine the invasiveness of the tumor cells. As is illustrated in FIG. 2, invasive cells cause a constriction of the gel raft that correlates with the degree of invasiveness of the cells seeded on the raft. Normal human fibroblasts, as a consequence of their normal biological functions, also result in constriction of the rafts upon which they are seeded.

FIG. 2A shows an example of gel contraction induced by melanoma cells of differing degrees of invasiveness. Duplicate experiments (top and bottom) with M619, MUM 2B, malignant melanoma cells derived from an eye melanoma (extreme left C918), another primary malanoma (M619 second from left), metastatic liver melanoma (MUM 2B 3rd from left), and non-aggressive and non-malignant primary melanoma (extreme right) OCM-1I cells. Only the malignant tumors constrict the gels. The degree of constriction correlates with the known degree of invasiveness of the cells seeded on the gel rafts.

FIG. 2B shows an example of gel constriction induced by ovarian carcinoma cells of different grades. The top row shows cells from a non-malignant ovarian biopsy placed on a floating raft and photographed over a 10 day period. The bottom row shows cells from a malignant ovarian carcinoma placed on a floating raft and photographed over a 10 day period. Malignant cells have constricted the gel raft while the size of the raft remains essentially unchanged in the presence of non-malignant cells.

### EXAMPLE 2

### The Use of an Unconstrained Gel Matrix to Assess the Invasiveness of Tumor Cells by Weight

Gel rafts were prepared and seeded with melanoma cells of known degrees of invasiveness as described in Example 1. Gel rafts upon which no cells were seeded were used as controls. At the end of the ten day incubation period, the gel rafts were removed from the 6-well plates; air dried; and weighed. Table 1 illustrates the loss in air dried gel weight caused by highly invasive and metastatic tumor cells.

TABLE 1
Gel Weight Loss

| Results (weights in | | | | | | |
|---|---|---|---|---|---|---|
| milligrams) Replicate | 1 | 2 | 3 | 4 | 5 | 6 |
| Control Gels | 93.67 | 92.67 | 91.00 | 99.00 | 77.33 | 94.00 |
| (collagen but no cells): | | | | | | |
| Non-invasive melanoma cells | 89.34 | 77.34 | 73.66 | 83.00 | 91.66 | 73.33 |
| (OCM1a): | | | | | | |
| Highly invasive primary | 56.33 | 57.00 | 55.33 | 59.00 | 54.67 | 53.00 |
| melanoma (M619): | | | | | | |
| Highly invasive metastatic | 53.67 | 46.67 | 53.00 | 65.38 | 57.67 | 57.33 |
| melanoma (MUM2b): | | | | | | |

These results indicate that the growth of invasive cells on a gel raft results in a decrease in the weight of the raft. The raft weight decreases with increasing invasiveness. Although normal human fibroblasts cause constriction of gel rafts, these cells do not cause the weight of the gel raft to decrease during cell growth.

### EXAMPLE 3

### The Use of an Unconstrained Gel Matrix to Assess the Invasiveness of Tumor Cells by Cell Growth Morphology

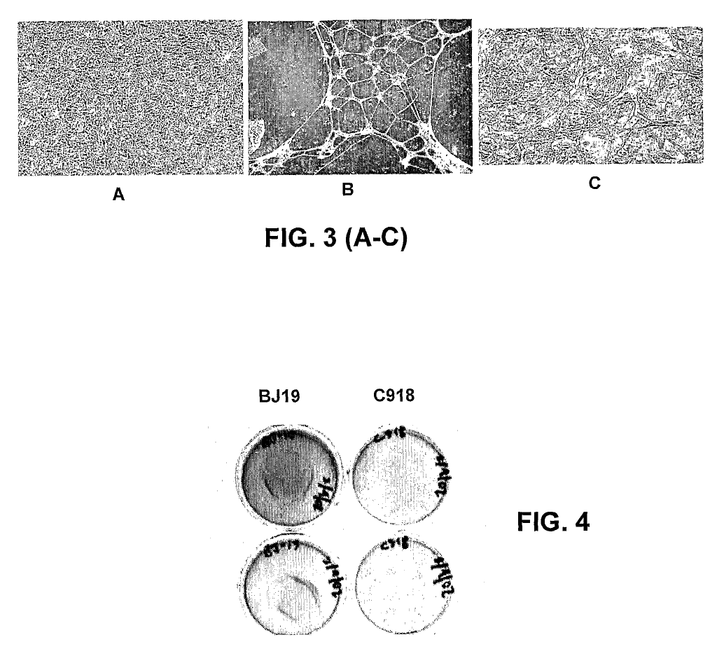

Cells grown on ECM layers that are greater than 100 microns in thickness assemble into structures that have different morphologies depending upon the invasiveness of the cells. Normal and non-invasive cells form sphere-like structures when grown under these conditions, while invasive cells form cord-like structures and, if the layer of ECM is sufficiently thick, highly invasive cells form spheroidal tumor nests consisting of tumor cells that are embedded in remodeled ECM protein. These tumor nests are permeated by non-vascular channels and display distinctive looping patterns of cell distribution. These different structural morphologies, which are illustrated in FIG. 3, can be readily observed and differentiated when cells grown on gel rafts are examined microscopically. Additional differentiation between highly invasive cells that form tumor nests and normal cells or cells having a lower degree of invasiveness can be obtained by staining the cells on the gel raft with a low molecular weight dextran (molecular weight range=70 KD-2,000,000 D) that has been labeled with a chromophore or fluorophore. Normal cells and cells having a low degree of invasiveness do not stain with such dextrans, whereas these dextrans can enter and stain the non-vascular channels of a tumor nest giving rise to visually distinctive staining patterns. Microscopic examination of the cell structures formed by cells grown on gel rafts can, therefore, provide confirmation of the degree of cellular invasiveness determined from gel constriction and/or weight loss.

### EXAMPLE 4

### The Use of an Unconstrained Gel Matrix to Screen Anti-Tumor Agents for Efficacy

Tumor cells in-vivo interact in significant ways with ECM proteins. This interaction has been reported to increase the resistance of invasive tumor cells to chemotherapeutic agents by as much as 150-fold. It is, therefore, desirable to assess the efficacy of anti-tumor agents on tumor cells grown in contact with ECM and further to assess the cytotoxicity of these same agents on normal cells under these same conditions.

As illustrated in Example 1, invasive tumor cells cause the constriction of unconstrained gel rafts. Normal fibroblasts cause a similar constriction of these rafts. In this method, the anti-tumor agent to be evaluated is added at an appropriate concentration to the medium upon which separate gel rafts, prepared as described in Example 1, that have been seeded, respectively, with the tumor cells of interest and with normal fibroblasts, have been prepared. Controls consist of identical rafts that are not exposed to the anti-tumor agent. As is illustrated in FIG. 4, an effective anti-tumor agent inhibits the constriction of those rafts seeded with tumor cells while not inhibiting the constriction of those rafts bearing normal fibroblasts. This indicates that the anti-tumor agent being evaluated is capable of suppressing the invasive behavior of the tumor cells, which is important in retarding the spread of a cancer, while not being cytotoxic to normal cells. This assessment, when carried out at several concentrations of the anti-tumor agent, permits the estimation of a suitable therapeutic level of the agent for a particular patient.

### EXAMPLE 5

### The Use of an Unconstrained Gel Matrix to Assess the Potential Immune Response of a Subject to Tumor Cells

Both the innate and adaptive immune systems provide defenses against cancers by mounting cellular and antibody responses directed against the tumor cells. Tumor cells have, however, evolved mechanisms for evading or preventing such immune responses.

Furthermore, many standard cancer therapies such as irradiation and chemotherapy can suppress immune responses in a patient. For this reason, it is desirable to be able to assess whether a patient's immune system is capable of mounting a response against the patients' tumor and, if so, the efficacy of this response, as part of the development of a therapeutic plan for the patient.

As illustrated in Example 1, invasive tumor cells cause the constriction of unconstrained gel rafts. This constriction can be inhibited by the binding of anti-tumor antibodies to the tumor cells; the destruction of tumor cells by cytotoxic lymphocytes; the secretion of cytokines by these lymphocytes; and interactive combinations of these. To assess the immune response to tumor cells, tumor cells from the patient are seeded on gel rafts prepared as described in Example 1. An appropriate patient specimen that is expected to contain immune effector agents is then applied to the tumor cells on the gel rafts. Suitable patient specimens for this purpose may include: whole blood or lymphatic fluid, which may contain both tumor-specific antibodies and cytotoxic lymphocytes; blood serum or plasma, which may contain tumor-specific antibodies; or peripheral mononuclear blood cell (PMBC) preparations, which may be enriched in cytotoxic lymphocytes. In the absence of tumor-responsive immune effectors in the patient specimens, invasive tumor cells cause constriction of the gel rafts as described in Example 1. This constriction is reduced if the patient specimen contains an immune effector that is active against the tumor cells. The magnitude of the reduction of constriction is indicative of the efficacy of those immune effectors that may be present in the patient specimen. Depletion of the patient specimen of antibodies and cytotoxic lymphocytes permits the detection of anti-tumor effects of cytokines and other chemical entities that may be present in the patient. The results of such an assessment provides the clinician with information and guidance with respect to estimating the potential clinical utility of including methods such as antibody therapies and the infusion of autologous cytotoxic lymphocytes in the treatment plan for a patient.

### EXAMPLE 6

### The Use of Inactivated Tumor Cells in an Unconstrained Gel Matrix to Stimulate an Anti-Tumor Response in a Subject

The dermal layer is the first level of active defense against infections in mammals. In support of this function, the body maintains high concentrations of cytotoxic lymphocytes, “memory “B” and “T” cells, antigen-presenting cells, and other components of the cellular and adaptive immune systems in immediate proximity to the dermis. This staging of immune system components at the dermal layer is the underlying basis for many “skin” tests for allergens and TB, and is also a route via which one can become sensitized against exogenous antigens.

### Materials and Methods

### Floating Rafts as Diagnostic Tools:

Malignant cells when placed on a “raft” of floating extracellular matrix molecules induce that raft of matrix to constrict over time if cells placed on it are malignant. Most non-malignant cells do not cause these matrices to constrict. Some types of normal fibroblasts also can induce these matrices to constrict, but it is the natural job of these fibroblasts in the normal non-diseased human body to remodel tissue, and these cells are used as control cells for testing drugs in parallel with tumor cells. Furthermore, the gels can be weighed to assess if they have been degraded by highly invasive tumor cells, because although fibroblasts constrict gels, they do not significantly decrease their weight.

Methods for producing the contracting gels shown in FIGS. 2A and B are as follows: Type I collagen was obtained from B&D Biosciences (Oak Park, Mass. 01730), and maintained at 4 degrees C. until use. 250 μl of liquid collagen was poured gently on the surface of 3 ml of 6 well (6×35 mm) plates containing EMEM (BioWhittalcer, Inc., Walkersville, Md.) supplemented with heat inactivated 15% fetal bovine serum (FBS, Fisher, Ontario, Canada) without the addition of exogenous ECM molecules or growth factors. After allowing ½ hour period of polymerization at 37 degrees C. in an incubator, the blank polymerized gels were removed and then seeded with cells.

### Seeding of Gels:

Primary uveal melanoma cell lines of low (OCM1a) and high (M619, C918) invasive potential, and a highly invasive metastatic uveal melanoma cell line (MUM2B) were then gently seeded onto the surfaces of the floating polymerized gels; the characteristics of these cells lines have been described in detail previously (Maniotis et al., 1999, 2002). Vascular channel formation by human uveal melanoma cells in vivo and in vitro.

Melanoma cells were plated at supersaturating cell densities of 1×10⁷ in 6 well (6×35 mm) plates. Gels that were not seeded were used as controls. Cells were grown for 10 days on the gels.

### Weighing of Gels:

24 approximately equal-sized pieces of weighing paper that labeled 1-24 were weighed, and the weight of each of the 24 pieces of paper was recorded. Each of 24 hydrated gels containing cells (18 gels were seeded and 6 were left blank) were placed onto the pre-weighed paper, completely dried, and then the dried gels plus the paper they had dried on were weighed, again. The difference in the weight of the paper alone, was subtracted from the weight of each weighing paper-gel combined weight. The gel assay was repeated six times. Each gel was weighed three times and the numbers below represent the mean of the weights. Data were analyzed using the parametric (t-test) as well as nonparametric (Kruskal Wallis test) methods. Individual tests show the following results:

### Averages of Control Gels (Collagen but No Cells)

93.67
92.67
91.00
99.00
77.33
94.00

### Averages of Non-Invasive Melanoma Gels OCM1a

89.34
77.34
73.66
83.00
91.66
73.33

### Average Weight of Highly Invasive Primary Melanoma M619

56.33
57.00
55.33
59.00
54.67
53.00

### Highly Invasive and Metastatic Melanomas MUM2b

53.67
46.67
53.00
65.38
57.67
57.33
Conclusions from this data include:
1. The blank gels are significantly different in weight from OCM1a (p=0.0374 for KW and p=0.0485 for t-test)
2. The blank gels are significantly different from MUM2b (p=0.0039 for KW and p<0.0001 for t-test)
3. The blank gels are significantly different from M619 (p=0.0039 for KW and p<0.0001 for t-test)
4. OCM1a is significantly different from MUM2b (p=0.0039 for KW and p=0.0003 for t-test)
5. OCM1a gels are significantly heavier than M619 (p=0.0039 for KW and p=0.0003 for t-test).
Tukey's test was found to generate data at the 95% level of significance:
Means: control=91.27, OCM1a=81.39 MUM2b=55.62, M619=55.89
Standard deviations: control=7.34, OCM1a=7.90 MUM2b=6.22, M619=2.06
The results of Tukey's test are:
Control and OCM1a are in one group while M619 and MUM2b are in another group, i.e., Control and OCM1a are not significantly different; control and MUM2b are significantly different, control and M619 are significantly different; OCM1a and MUM2b are significantly different; OCM1a and MUM2b are significantly different. M619 and MUM2b are not significantly different.

### Floating Rafts as Toxicity and Efficacy Tools for Chemotherapy:

Tumor cells in contact with extracellular matrix resist chemotherapeutic agents. Because the gel is composed of ECM components, the same rafts can be used in vitro as a more accurate tool to test new chemotherapeutic drugs. Gel constriction constitutes a functional assay to evaluate cell resistance to the effects of drugs, the results of which can be verified using Trypan Blue (to measure cell death). Aggressive cells are as much as 150 times less-resistant to drugs when the cultures are grown in the presence of matrix or when cells are grown as aggregates, reflecting the typically observed clinical resistance of malignant cells in a more normal tissue environment. However, normal fibroblasts, although they are not malignant or cancerous, are known to constrict gels because it is their normal function in the body to remodel tissues. Therefore, the ability of a normal cell type (fibroblasts) can be compared with the known ability of malignant tumor cells (melanoma, ovarian carcinoma, lung, kidney, bladder, hepatocellular carcinoma, pheochromocytomas, oral cancers, and others) to assess a drugs' toxicity (if the fibroblasts are inhibited from contracting the gel a drug is considered toxic) at the same time as drug efficacy is determined on blocking tumor cell-induced gel constriction, by using these floating matrix “rafts.” Agents that are potentially capable of blocking gel constriction induced by highly invasive tumor cells are tested with normal cells whose job it is to normally remodel matrices, and with tumor cells of varying grades. Drugs, agents, or strategies for specifically blocking aggressive tumor cells must pass the constricting gel assay-if the drug kills normal fibroblastic cells, or blocks their ability to constrict the floating gels (fibroblasts, endothelial cells, kidney cells), the chemotherapeutic compound or regimen is not pursued further. However, if a particular compound or regimen does not block the constriction of gels by fibroblasts, endothelial cells or kidney cells, yet blocks malignant tumor cell gel constriction at the same dose, the compound or regimen is tested in other even more stringent assays.

FIG. 4 illustrates effects of an anti-tumor compound on melanoma cells. Example of melanoma cells (C918-highly invasive primary melanoma cells blocked from constricting gels-two right dishes) because of treatment with an experimental anti-tumor compound (note no constriction has occurred) compared to BJ 19 normal human fibroblasts (note that they are beginning to constrict the left 2 gels. This drug would be a good non-toxic yet effective candidate to begin pre-clinical testing.

Methods for producing contracting gels shown in FIG. 2 were identical to those methods described for the constricting assay shown in FIG. 4 only a drug was added to all the cultures for a predetermined period and concentration.

### Therapeutic Patch:

Matrix gels can be fashioned using recombinant thrombin added to a patient's own serum (fibrinogen) obtained during biopsy procurement. In this context, the gel can be used to diagnose cancer, but then if the gel constricts indicating the tumor cells on it are malignant after drying it can be used as an in vivo immunopatch, by placing it in contact with exposed dermis to study the immunorejection of that patient's (animal's) own tumor cells. Inactivated tumor cells may be used to stimulate an immune response.

### DOCUMENTS

Berube M., et al., Role of the extracellular matrix proteins in the resistance of SP6.5 uveal melanoma cells toward cisplatin. Int. J. Oncol. 26 (2):405-413, (2005).
Stein, Ruth A, et al. Coordinate changes in drug resistance and drug-induced conformational transitions in altered-function mutants of the multidrug transporter P-glycoprotein. Biochemistry, April 10; 40(14):4332-9, (2001).
Maniotis, A. J., et al. Control of Melanoma Morphogenesis Endothelial Survival, and Perfusion By Extracellular Matrix. Lab Investigation. Vol. 82 No. 8 p. 1083-1092, 2002.
Ethicon Endo-Surgery, Inc. Mammotone, Touch Briefings (http://www.bbriefings.com/pdf/952/Ethicon_{—}2_tech.pdf)
Cancer Treatment Centers of America—Diagnostic Tools (http://www.cancercenter.com/diagnostic-tools.cfm)
Shirakawa K, et al. in Vasculogenic Mimicry and Angiogenesis of Inflammatory Breast Cancer Xenograft. Cancer Research 62, 560-566, Jan. 15, 2002.
Maniotis, A. J. et al. Chromatin Organization Measured By Alu Restriction Enzyme Changes With Malignancy, And Is Regulated By The Extracellular Matrix And Cytoskeleton. American Journal Of Pathology, Vol 166, No 4, April (2005).
Maniotis, A. J. et al. Vascular Channel Formation By Human Uveal Melanoma Cells In Vivo and In Vitro: Vasculogenic Mimicry. Amer. J. Path. Vol. I55, No 3, 739-752, September (1999, 2002)

1. A method to determine invasive potential of tumor cells using a matrix gel assay, the method comprising:
(a) providing a first and second matrix support comprising at least one component of the extracellular matrix (ECM) suspended in a cell culture medium;
(b) seeding the first matrix support with tumor cells and the second matrix support with control cells;
(c) providing conditions for the tumor cells to grow in the first matrix support and the control cells to grow in the second matrix support;
(d) preparing a dried sample of the matrix support;
(e) comparing the dried weight of the first matrix of the tumor cells to the dried weight of the second matrix of the control cells; and
(f) determining the invasive potential of the tumor cells by comparing the dried weight of the first matrix with tumor cells and the dried weight of the second matrix with control cells.

2. A method to determine invasive potential of tumor cells using a matrix gel assay, the method comprising:
(a) providing a first and second matrix support comprising at least one component of the extracellular matrix (ECM) suspended in a cell culture medium;
(b) seeding the first matrix support with tumor cells;
(c) providing conditions for the tumor cells to grow in the first matrix support;
(d) preparing a dried sample of the first and the second matrix supports;
(e) comparing the dried weight of the first matrix of the tumor cells to the dried weight of the second matrix; and
(f) determining the invasive potential of the tumor cells by comparing the dried weight of the first matrix with tumor cells and the dried weight of the second matrix.

3. The method of claim 1, wherein the tumor cells are melanoma cells.

4. The method of claim 1, wherein the tumor cells are seeded to a saturation density.

5. The method of claim 1, wherein the matrix support is air-dried.

6. A method to screen for a potential anti-tumor agent using a matrix gel assay, the method comprising:
(a) providing a first and second matrix support comprising at least one component of the extracellular matrix (ECM) suspended in a cell culture medium, wherein the first and second matrix supports are seeded with tumor cells;
(b) contacting the first matrix support with the candidate anti-tumor agent;
(c) providing conditions for growth of tumor cells in the matrix supports;
(d) preparing a dried sample of the matrix supports;
(e) comparing the weight of the first matrix with the tumor cells to the dried weight of the second matrix of the tumor cells; and
(f) determining that the candidate anti-tumor agent is an antitumor agent if the dried weight of the first matrix with tumor cells is significantly more than the dried weight of the second matrix with tumor cells.

7. A method to screen for a potential anti-tumor agent using a matrix gel assay, the method comprising:
(a) providing a first and second matrix support comprising at least one component of the extracellular matrix (ECM) suspended in a cell culture medium, wherein the first matrix support is seeded with tumor cells and second matrix support is seeded with non-tumor control cells;
(b) contacting the first and second matrix supports with the candidate anti-tumor agent;
(c) providing conditions for growth of tumor cells and control cells in the matrix supports;
(d) preparing a dried sample of the matrix supports;
(e) comparing the weight of the first and second matrix supports contacted with the candidate anti-tumor agent to the dried weight of the first and second matrix supports not contacted with the candidate anti-tumor agent; and
(f) determining that the candidate anti-tumor agent is an anti-tumor agent if the dried weight of the first matrix with tumor cells contacted with the candidate anti-tumor agent is significantly more than the dried weight of the first matrix with tumor cells not contacted with the candidate anti-tumor agent and the dried weight of the second matrix support with control cells contacted with the candidate anti-tumor agent is similar to the dried weight of the second matrix support with control cells not contacted with the candidate anti-tumor agent.

8. The method of claim 7, wherein the control cells are fibroblasts.

9. A method to stimulate immune response against tumor cells, the method comprising:
(a) obtaining a matrix support comprising seeded tumor cells, wherein the matrix and the cells are in a dried condition;
(b) administering the dried matrix support with tumor cells as a dermal patch to stimulate an immune response; and
(c) monitoring the immune response against the tumor cells.

10. A therapeutic patch comprising:
(a) a first layer comprising a matrix support comprising at least one component of the extracellular matrix seeded with tumor cells, wherein the matrix support is in a dried condition; and
(b) a second layer comprising fibrin and thrombin, wherein the second layer is adapted to be applied as a dermal patch.

11. The therapeutic patch of claim 10, wherein the fibrin is derived from fibrinogen obtained from a serum sample of an individual.

12. The therapeutic patch of claim 10, wherein the thrombin is a recombinant thrombin.

13. The method of claim 2, wherein the tumor cells are melanoma cells.

14. The method of claim 2, wherein the tumor cells are seeded to a saturation density.

15. The use method of claim 2, wherein the matrix support is air-dried.
